(19) United States
(12) Patent Application Publication
(10) Pub. No.: US 2018/0177706 A1
(43) Pub. Date: Jun. 28, 2018

(54) COMPOSITIONS CONTAINING HYALURONIC ACID AND SYNERGISTIC ANTI-HYALURONIDASE ACTIVES

(71) Applicant: L'OREAL, Paris (FR)

(72) Inventors: Zhi PAN, Clark, NJ (US); Anne-Laure Suzanne BERNARD, Clark, NJ (US); Hy Si BUI, Clark, NJ (US); Fan HU, Clark, NJ (US)

(21) Appl. No.: 15/390,220

(22) Filed: Dec. 23, 2016

(51) Int. Cl. A61K 8/73; A61K 8/97; A61K 8/49; A61K 9/00

(52) U.S. Cl. CPC A61K 8/735; A61K 8/97; A61K 9/0019; A61K 9/0014; A61K 8/4993

(57) ABSTRACT

Compositions comprising a hyaluronic acid adjuvant system comprising hyaluronic acid, purple rice extract and dipotassium glycyrrhizate, present together in amounts sufficient to produce synergistic anti-hyaluronidase activity, and provided for cosmetic and other uses.

### CROSS-REFERENCE TO RELATED APPLICATIONS

This patent application claims benefit of U.S. patent application Ser. No. 15/056,710, filed on Feb. 26, 2016, entitled “STABLE EMULSIFIED GEL COMPOSITION HAVING A HIGH CONCENTRATION OF ACTIVE COMPONENTS,” the disclosure of which is incorporated by reference as if fully rewritten herein.

### FIELD OF THE INVENTION

The present invention is directed to compositions having active components for application to keratinous tissues, particularly skin. More specifically, the present invention is directed to compositions having a hyaluronic acid delivery system including hyaluronic acid and a combination of hyaluronidase inhibitors that work synergistically, the compositions delivered in various formulations for delivery by various modes including, but not limited to, topical, patch and injection.

### BACKGROUND OF THE INVENTION

Hyaluronic acid (HA) is a natural mucopolysaccharide formed of alternating units of D-glucuronic acid and N-acetylglucosamine in a linear chain. HA is found both the dermis and the epidermis of the skin, where it has a protective, structure stabilizing and shock-absorbing function. HA is an important component of the skin matrix that serves as a connective tissue supporter and water retaining substance. HA that is in the skin, and as exogenously applied, plays a critical role in connective tissue for achieving skin's fuller, firmer and more youthful appearance.

Exogenously applied HA can penetrate the skin and provide supplemental moisturizing and viscoelastic properties to the skin, making the skin appear softened and restoring its elasticity, and thus, confers an anti-wrinkle effect. Delivery of HA into skin is a critical anti-aging strategy, and HA is a common ingredient in skin care formulations. But HA has very short life in skin (<1 day) due to the presence of hyaluronidase (HAase) in the skin tissue. HA fragments resulting from degradation of HA are not efficient to provide longer lasting benefits through cosmetic treatments.

There remains a need to provide a composition, including skin care compositions, capable of stably carrying one or more agents, with or without hyaluronic acid, that provide protection against the effects of hyaluronidase, such compositions for delivery to skin tissue to one or more of confer, enhance and extend the benefits of endogenous HA and of HA treatments.

### BRIEF SUMMARY OF THE INVENTION

The invention provides, in various embodiments, compositions formulated for various modes of delivery, the compositions comprising a plurality of components to one or more of confer, enhance and extend the benefits of endogenous HA and of HA treatments. The compositions are adjuvants for one or more of exogenously delivered HA and endogenous HA.

In some embodiments, the invention provides compositions comprising (a) HA, (b) purple rice extract, and (c) dipotassium glycyrrhizate, present in the compositions in amounts sufficient to produce synergistic anti HAase activity.

In some embodiments, the invention provides compositions comprising (a) HA provided in a solid or semi-solid soluble or swellable form, (b) purple rice extract, and (c) dipotassium glycyrrhizate, present in the compositions in amounts sufficient to produce synergistic anti HAase activity, wherein the solid or semi-solid soluble or swellable HA is in a form selected from nano and micron scale needles, and wherein purple rice extract and dipotassium glycyrrhizate are present in fluid, solid or semi-solid soluble or swellable form.

In accordance with some embodiments, the compositions include (a) about 0.01% to about 20% of HA, (b) about 0.01% to about 20% of purple rice extract; and (c) about 0.01% to about 20% of dipotassium glycyrrhizate, all amounts present as percentages by weight based on the total weight of the composition. In accordance with some specific embodiments, the formulation is suitable for application to a keratinous substrate for topical application in gel, cream, or serum form.

In accordance with some specific embodiments, the compositions include (a) about 0.01% to about 20% of HA, (b) about 0.01% to about 20% of purple rice extract; and (c) about 0.01% to about 20% of dipotassium glycyrrhizate, all amounts present as percentages by weight based on the total weight of the composition, and wherein the purple rice extract and dipotassium glycyrrhizate are present in a ratio of 1:1.

In accordance with some specific embodiments, the compositions include (a) about at least about 3% of HA, (b) about up to about 3% of purple rice extract; and (c) about up to about 5% of dipotassium glycyrrhizate, all amounts present as percentages by weight based on the total weight of the composition.

In accordance with some specific embodiments, the compositions include (a) about 0.01% to about 3% of HA, (b) about 0.01% to about 3% of purple rice extract; and (c) about 0.01% to about 5% of dipotassium glycyrrhizate, all amounts present as percentages by weight based on the total weight of the composition.

In an exemplary embodiment, a composition is in the form of a skin care composition for topical application. The composition includes a hyaluronic acid delivery component. The hyaluronic acid delivery component includes a combination of active ingredients. The combination of active ingredients includes each of from about 0.01 to about 20% by weight, and more particularly (a) from about 0.01% to about 3% of HA, (b) from about 0.01% to about 3% of purple rice extract; and (c) from about 0.01% to about 5% of dipotassium glycyrrhizate.

In accordance with some embodiments, the compositions include (a) about 0.01% to about 20% of HA, (b) about 0.01% to about 20% of purple rice extract; and (c) about 0.01% to about 20% of dipotassium glycyrrhizate, all amounts present as percentages by weight based on the total weight of the composition. In accordance with some specific embodiments, the formulation is suitable for application in injectable form.

In accordance with some specific embodiments, the compositions include (a) about 0.01% to about 3% of HA, (b) about 0.001% to about 0.5% of purple rice extract; and (c) about 0.001% to about 0.5% of dipotassium glycyrrhizate, all amounts present as percentages by weight based on the total weight of the composition. In accordance with some specific embodiments, the formulation is suitable for application in injectable form.

In accordance with some embodiments, the compositions include (a) about 50% to about 100% of HA in solid or semi-solid soluble or swellable form, (b) about 0.001% to about 3% of purple rice extract; and (c) about 0.001% to about 5% of dipotassium glycyrrhizate, all amounts present as percentages by weight based on the total weight of the composition. In accordance with some specific embodiments, the purple rice extract and the dipotassium glycyrrhizate are present in one of fluid, solid or semi-solid soluble or swellable form.

In accordance with some specific embodiments, the compositions include (a) about 80% to about 100% of HA in solid or semi-solid soluble or swellable form, (b) about 0.001% to about 0.5% of purple rice extract; and (c) about 0.001% to about 0.5% of dipotassium glycyrrhizate, all amounts present as percentages by weight based on the total weight of the composition. In accordance with some specific embodiments, the purple rice extract and the dipotassium glycyrrhizate are present in one of fluid, solid or semi-solid soluble or swellable form.

Another aspect of the invention provides methods for preparing compositions comprising HA, purple rice extract, and dipotassium glycyrrhizate.

A further aspect of the invention provides methods for preparing an cosmetic formulation comprising an HA delivery system, the method comprising the step of including in said formulation (a) about 0.01% to about 20% of HA, (b) about 0.01% to about 20% of purple rice extract; and (c) about 0.01% to about 20% of dipotassium glycyrrhizate, and one or more components for forming one of an aqueous serum, an oil-in-water emulsion, and a water-in-oil emulsion.

The present disclosure is also directed to a method for cosmetic treatment of keratinous tissues by applying an above-disclosed composition to keratinous tissue, the methods of application selected from direct topical application of the compositions, topical application effected by use of one or more components in fluid, solid or semi-solid soluble or swellable nano- or micro-needle form, and injectable form.

Other features and advantages of the present invention will be apparent from the following more detailed description of the preferred embodiment which illustrates, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

FIG. 1 shows a table representing the results of a dose screening in vitro assay of various known HAase inhibitors;

FIG. 2 shows a graph of the loss of viscosity in an HA gel model for assessing HAase inhibition; and,

FIG. 3 shows a bar graph representing the calculated inhibition factor for individual and combined HAase inhibitors.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The term “comprising” (and its grammatical variations) as used herein is used in the inclusive sense of “having” or “including” and not in the exclusive sense of the terms “consisting only of,” “consisting essentially of” and “consisting of.”

“Cosmetically acceptable” means compatible with any keratinous tissue. For example, “cosmetically acceptable carrier” means a carrier that is compatible with any keratinous tissue.

“Homogenous” means having the visual appearance of being substantially uniform throughout, i.e., visually appears as a single phase emulsion.

“Keratinous tissue,” as used herein, includes, but is not limited to, skin, hair, and nails.

“W/O emulsion,” and “W/Si emulsion” as used herein, includes a water phase dispersed in an oil phase, where the oil phase is a continuous phase and includes at least one Si emulsifier.

Synergistic activity is present when a measured effect with a combination of actives is significantly larger than the expected value based on the sum of the activities of the individual components. Significantly larger than the expected value refers to measured anti HAase activities that are at least 25% greater than expected values. Some of the compositions as shown in the examples exhibit synergistic anti HAase activity greater than 25% over expected values.

It has been unpredictably discovered by the inventors that the combination of hyaluronic acid with two discrete hyaluronidase inhibitors, purple rice extract and dipotassium glycyrrhizate, provides extended stability and resistance of HA to hyaluronidase. As disclosed herein, when in combination with HA, purple rice extract and dipotassium glycyrrhizate are dramatically stronger than the additive effects of the individual compounds alone. This surprising synergistic effect, in combination with the other known benefits of the compounds individually, can be employed advantageously in compositions for application to keratinous tissues, particularly skin, to provide anti-aging and other related benefits. Compositions having HA delivery systems that include HA and mixtures of purple rice extract and dipotassium glycyrrhizate are more fully described herein below, as are other optional components, and are useful in cosmetic formulations.

The compositions according to the present disclosure include HA, purple rice extract and dipotassium glycyrrhizate, which may be, in some embodiments, provided in an emulsified gel system comprising an emulsifier and one or more hyaluronic acid delivery components that include HA, purple rice extract and dipotassium glycyrrhizate.

Hyaluronic Acid Delivery Component

The hyaluronic acid delivery component includes a combination of HA, purple rice extract and dipotassium glycyrrhizate. These combinations provides HA benefits including moisturizing of the skin upon application and allows the delivery and absorption of hyaluronic acid.

Hyaluronic Acid

“Hyaluronic acid,” HA, also known as “hyaluronan” and “hyaluronate”, as used herein, relates to polymers of disaccharides composed of D-glucuronic acid and D-N-acetylglucosamine, linked together via alternating β-1,4 and β-1,3 glycosidic bonds. Hyaluronan can be 25,000 disaccharide repeats in length and range in size from 5,000 to 20,000,000 Da in vivo. The average molecular weight in human synovial fluid is 3-4 million Da. The hyaluronic acid according to the present disclosure includes salts and derivatives of hyaluronic acid, which can be crosslinked or non-crosslinked.

HA is present in a composition according to the disclosure in a final weight percent amount that is determined as the product of the percentage purity of the HA in the raw material and the percentage of the raw material used in the formulation. In accordance with some embodiments, HA is present in the compositions according to the disclosure in amounts that range from about 0.01% to about 20%, and in some embodiments from about 0.1% to about 10%, and in some further embodiments from about 0.5% to about 5%. In some representative embodiments, HA is present from about 0.5% to about 3%, and from about 1% to about 2%. In yet other embodiments, HA is present up to about 3%. And in yet other embodiments, HA is present from at least about 1%, or at least about 2%, or at least about 3%, and up to about 20%. In some specific embodiments, HA is present at about 1.3%. Certain representative embodiments include compositions for topical or injectable application.

Thus, HA may be present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

In some embodiments, HA is present in the compositions according to the disclosure in amounts that range from about 40% to about 100%, and in some embodiments from about 80% to about 100%, and in some further embodiments from about 90% to about 100%. In some representative embodiments, HA is present from about 50% to about 75%, and from about 60% to about 70%. In yet other embodiments, HA is present up to about 100%. And in yet other embodiments, HA is present from at least about 40%, or at least about 50%, or at least about 60%, or at least about 80% and up to about 100%. In some specific embodiments, HA is present at about 90%. Certain representative embodiments include compositions that are one or more of fluid, solid or semi-solid soluble or swellable form, in some embodiments in the form of microneedles or nanoneedles.

Thus, HA may be present from about 40, 45, 50, 55, 60, 65, 70, 75, 80, 85, 90, 95, to about 100 percent by weight, including increments and ranges therein and there between.

Hyaluronidase Inhibitor

HA inhibitors having a synergistic anti-HAase effect according to the disclosure include purple rice extract (e.g., oryza sativa (rice) extract), and dipotassium glycyrrhizate, including derivatives thereof.

In some embodiments, the compositions may comprise one or more supplemental HA inhibitors, including, but not limited to, cucumis sativus fruit, terminalia catappa, ascorbic acid, mushroom stem extract, ganoderma cucidum extract and combinations thereof.

In accordance with the various embodiments, the compositions comprise purple rice extract.

Purple rice extract is present in a composition according to the disclosure in a final weight percent amount that is determined as the product of the percentage purity of the purple rice extract in the raw material and the percentage of the raw material used in the formulation. Purple rice extract is present in the compositions according to the disclosure in amounts that range from about 0.01% to about 20%, and in some embodiments from about 0.1% to about 10%, and in some further embodiments from about 0.5% to about 5%. In some representative embodiments, purple rice extract is present from about 0.5% to about 3%, and from about 1% to about 3%. In yet other embodiments, purple rice extract is present up to about 3%. And in yet other embodiments, purple rice extract is present from at least about 1%, or at least about 2%, or at least about 3%, and up to about 20%. In some specific embodiments, purple rice extract is present at about 1.2%. Certain representative embodiments include compositions for topical application.

Thus, purple rice extract may be present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

In some embodiments, for example for injectable application, purple rice extract is present in the compositions according to the disclosure in amounts that range from about 0.001% to about 3%, and in some embodiments from about 0.001% to about 2%, and in some further embodiments from about 0.01% to about 1%. In some representative embodiments, purple rice extract is present from about 0.01% to about 0.5%, and from about 0.1% to about 0.5%. In yet other embodiments, purple rice extract is present up to about 3%. And in yet other embodiments, purple rice extract is present from at least about 0.01%, or at least about 0.1%, or at least about 0.5%, and to up to about 3%. In some specific embodiments, purple rice extract is present at about 0.5%. Certain representative embodiments include compositions for injectable application.

Thus, purple rice extract may be present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0 to about 3.0 percent by weight, including increments and ranges therein and there between.

In some embodiments, purple rice extract is present in a fluid form, for example, but not limited to, a gel, cream, lotion, serum, spray, and injectable. In other embodiments, purple rice extract is present in solid or semi-solid soluble or swellable form, for example in the form of nano- or micron-scale needles.

In accordance with the various embodiments, the compositions comprise dipotassium glycyrrhizate. The dipotassium glycyrrhizate according to the present disclosure includes salts and derivatives of dipotassium glycyrrhizate.

Dipotassium glycyrrhizate is present in a composition according to the disclosure in a final weight percent amount that is determined as the product of the percentage purity of the dipotassium glycyrrhizate in the raw material and the percentage of the raw material used in the formulation. Dipotassium glycyrrhizate is present in the compositions according to the disclosure in amounts that range from about 0.01% to about 20%, and in some embodiments from about 0.1% to about 10%, and in some further embodiments from about 0.5% to about 8%. In some representative embodiments, dipotassium glycyrrhizate is present from about 0.5% to about 5%, and from about 1% to about 5%. In yet other embodiments, dipotassium glycyrrhizate is present up to about 5%. And in yet other embodiments, dipotassium glycyrrhizate is present from at least about 1%, or at least about 2%, or at least about 3%, or at least about 4%, or at least about 5%, and up to about 20%. In some specific embodiments, dipotassium glycyrrhizate is present at about 2%. Certain representative embodiments include compositions for topical application.

Thus, dipotassium glycyrrhizate may be present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

In some embodiments, for example for injectable application, dipotassium glycyrrhizate is present in the compositions according to the disclosure in amounts that range from about 0.001% to about 5%, and in some embodiments from about 0.001% to about 3%, and in some further embodiments from about 0.01% to about 1%. In some representative embodiments, dipotassium glycyrrhizate is present from about 0.01% to about 0.5%, and from about 0.1% to about 0.5%. In yet other embodiments, dipotassium glycyrrhizate is present up to about 5%. And in yet other embodiments, dipotassium glycyrrhizate is present from at least about 0.01%, or at least about 0.1%, or at least about 0.5%, and to up to about 5%. In some specific embodiments, dipotassium glycyrrhizate is present at about 0.5%. Certain representative embodiments include compositions for injectable application.

Thus, dipotassium glycyrrhizate may be present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0 to about 5.0 percent by weight, including increments and ranges therein and there between.

In some embodiments, dipotassium glycyrrhizate is present in a fluid form, for example, but not limited to, a gel, cream, lotion, serum, spray, and injectable. In other embodiments, dipotassium glycyrrhizate is present in solid or semi-solid soluble or swellable form, for example in the form of nano- or micron-scale needles.

In various embodiments, each of the inhibitors purple rice extract and dipotassium glycyrrhizate is present in the compositions in the foregoing amounts, wherein the ratio of purple rice extract to dipotassium glycyrrhizate ranges from about 2:1 to 1:2. In some exemplary embodiments, purple rice extract and dipotassium glycyrrhizate are present in a ratio of about 1:1.

Compositions Comprising Combinations of Ha and Hyaluronidase Inhibitors

In the various embodiments, one or more of HA and HAase inhibitors is present in a composition according to the invention in a fluid form, for example, but not limited to, a gel, cream, lotion, serum, spray, and injectable. In other embodiments, one or more of HA and HAase inhibitors is present in solid or semi-solid soluble or swellable form, for example in the form of nano- or micron-scale needles.

Generally, transdermal needle delivery may be achieved using nano or micron-scale needles to deliver active ingredients to a specific layer of skin, which is composed of stratum corneum (<20 ρm), epidermis (<100 μm), and dermis (300 to 2,500 μm). Delivery may be in a topically applied form, in a solid patch form, in an injectable form, or other forms known in the art. Thus, in some embodiments, the needles may be applied in a formulation comprising of one or more of HA and HAase inhibitors, the additional ingredients, selected from but not limited to, HAase inhibitors selected from purple rice extract and dipotassium glycyrrhizate, and other additives and adjuvants, wherein the additional ingredients may also be in solid or semi-solid needle form, or in a solubilized or suspension form.

In accordance with the instant disclosure, needles may be formed of 100% active in solid or semi-solid form, the needles having a general conical shape. Microneedles may thus have a top diameter of approximately 30 μm, an effective length of 100 to 500 μm, and a hardness selected to suitably penetrate the targeted layer of skin. In addition, to deliver active ingredients by soluble microneedles, the delivery medium for the needles will be free from any solvent, such as water, or other components to enable solvation at the delivery target. Micron and nano-needles and their methods of preparation are generally known in the art.

According to the various embodiments, the compositions comprising one or a plurality of HA and HAase inhibitors may be applied topically, topically in the form of micro or nano-needles, by injection, or combinations of these. Thus, in some representative embodiments, the compositions are topical formulations in the form of a gel, cream, lotion, serum, spray, wherein the components are provided in solution, suspension or emulsion. In other embodiments, the compositions are topical formulations in the form of a gel, cream, lotion, serum, spray, wherein one or more of the components, for example, HA, is present in solid or semi-solid soluble or swellable form, for example in micro-needle form. In some specific embodiments, the compositions are provided in separate containers for either separate sequential or combined application.

In some embodiments, the compositions are provided in a patch or other solid delivery form. In some embodiments, the compositions may be administered by injection. According to some such embodiments, the injectable may be selected from one or a combination of HA and HAase inhibitors, optionally with other ingredients.

Adjuvants

In a known manner, compositions according to present disclosure may also contain adjuvants that are common in cosmetics, such as humectants, preserving agents, antioxidants, complexing agents, solvents, fragrances, bactericides, odour absorbers, vitamins, moisturizers, self-tanning compounds and antiwrinkle active agents. The amounts of these various adjuvants are those conventionally used in the field under consideration, for example from 0.01% to 20% of the total weight of the composition. Depending on their nature, these adjuvants may be introduced into the fatty phase, into the aqueous phase and/or into lipid vesicles.

Other adjuvants suitable for use with the emulsified gel according to the present disclosure include, for example, water-soluble or water-miscible solvents or co-solvents or oil-soluble or oil-miscible solvents or co-solvents. Suitable examples of additives and adjuvants include, but are not limited to, fatty alcohols, fatty amides, alkylene carbonates, glycols, lower alcohols (e.g. ethanol, propanediol), dispersion enhancing agents, polymers, thickening agents, stabilizers, moisturizers, humectants, colorants, fillers, chelating agents, antioxidants (e.g. BHT, tocopherol), essential oils, fragrances, dyes, neutralizing or pH-adjusting agents (e.g., citric acid, triethylamine (TEA) and sodium hydroxide), preservatives, bactericides, conditioning or softening agents (e.g., panthenol and allantoin), extracts, such as botanical extracts, or any other ingredient commonly used in cosmetics for this type of application. Additives and adjuvants may be present in the compositions in amounts generally ranging from about 0.01% to about 10% by weight. Examples of cosmetic active agents or dermatological active agents include free-radical scavengers, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in any water phase(s) or oil phase(s) that is/are present in the sunscreen composition (i.e., aqueous and/or fatty (oil) phase).

Of course, a person skilled in the art will take care to choose this or these optional additional compounds so that the advantageous properties intrinsically attached to the composition in accordance with the present disclosure are not, or not substantially, detrimentally affected by the envisaged addition or additions.

A person skilled in the art will take care to select this or of these optional additional compound(s), and/or the amount thereof, such that the advantageous properties of the composition according to the present disclosure are not, or are not substantially, adversely affected by the envisaged addition.

Compositions according to the present disclosure finds its application in a wide variety of treatments, especially cosmetic treatments, of the keratinous tissue, such as skin, the lips and the hair, including the scalp, especially for treating, protecting or caring for the skin, the lips and/or the hair, and/or for making up the skin and/or the lips. It may also be intended for treating dry skin and/or dry lips, while at the same time delivering moisturization.

Methods

Compositions according to the present disclosure is prepared by combining the water and oil phase ingredients, including the hyaluronic acid delivery component, in a vessel and heating this water phase while mixing additional phases to form a homogeneous phase. The heating includes heating to a temperature from about 25° C. to 50° C. or about 35° C. The phase ingredients were premixed, when appropriate. The homogenization is performed for a time that does not exceed 2 minutes. If necessary, the pH is adjusted to desired level.

The following examples are intended to further illustrate the present invention. They are not intended to limit the invention in any way. Unless otherwise indicated, all parts are by weight.

### EXAMPLES

### Example 1: In Vitro Demonstration of Inhibition

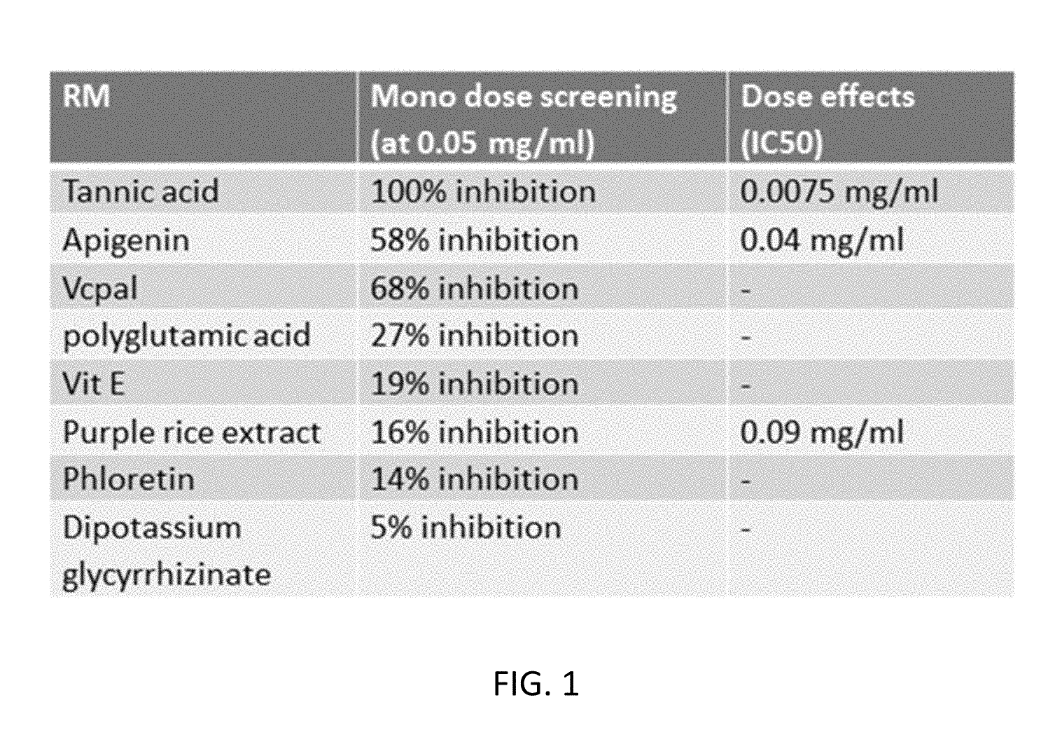

Antioxidants having known hyaluronidase inhibition properties were tested in vitro to establish baseline inhibition. Referring now to the drawings, selected results are shown in the table of FIG. 1, with the known HAase inhibitor Apigenin as a positive control. While tannic acid was a very potent inhibitor of HAase, it was shown to be highly insoluble and raised formulation challenges. Purple rice extract and dipotassium glycyrrhizate were each identified as mild inhibitors of HAase in this assay at a concentration of 0.05 mg/mL. Dose effect study showed that purple rice extract is able to reach IC 50 at about 0.09 mg/ml, while Dipotassium glycyrrhizate has no clear dose effect up to testing dose of 0.5 mg/ml.

As shown in FIG. 1, the in vitro test also demonstrated unexpected synergy between purple rice extract and Dipotassium glycyrrhizate, for example in a representative composition comprising 0.05 mg/ml of each ingredient (i.e., at ratio of 1:1).

Referring again to FIG. 1, the results show that the measured inhibition of the combination of purple rice extract and Dipotassium glycyrrhizate is surprisingly greater than the sum of the individual measured inhibition results, wherein the measured inhibition was 44%, while the sum of the individual results was 35%, representing an increase of ˜26%.

### Example 2: In Vitro Testing of Inhibition

Hyaluronidase inhibition activity was evaluated in a HA gel model of HAase inhibition-based kinetic viscoelastic profile measurement. According to the experimental design, various individual controls and known HAase inhibitors were tested (alone and in combination). In this model, HAase was introduced into a gel composed of HA. Upon exposure, the HAase decomposes the HA gel in a time dependent manner which is directly correlated to the viscosity of the HA gel. Test compounds were introduced to the HA gel system and degree of HAase inhibition as gauged by measuring viscosity (change in viscosity) over time.

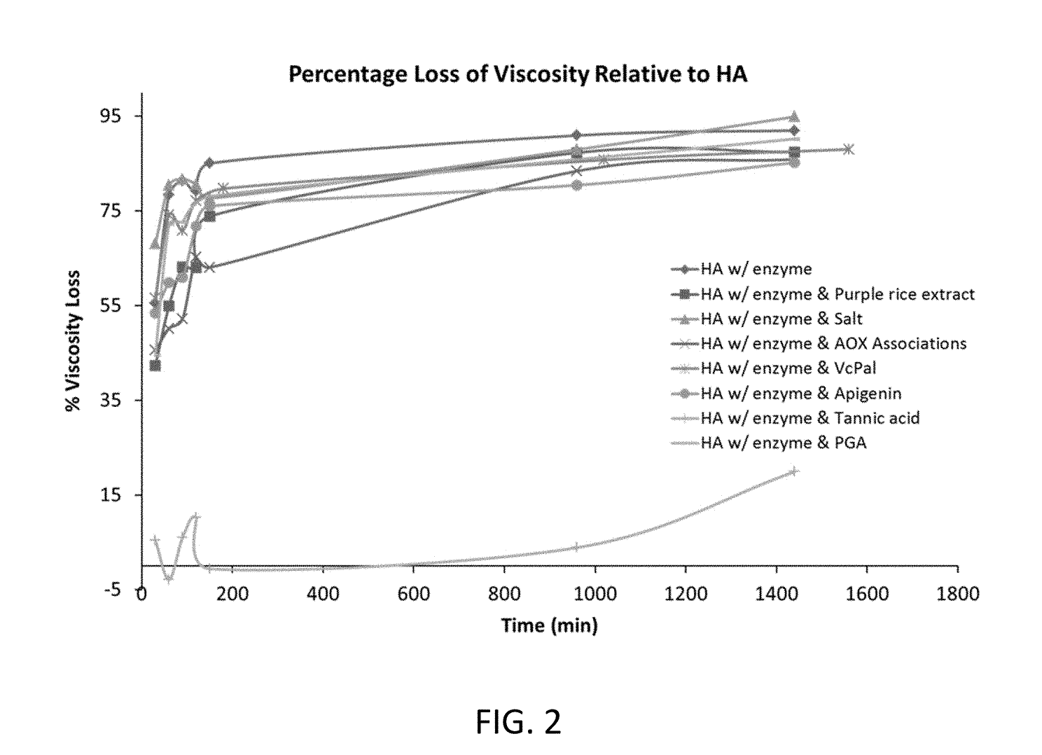

At each time point, % of Viscosity Loss is compared to the viscosity of HA gel without enzyme. Referring now to FIG. 2, the results show that, with the exception of Tannic acid, the association of purple rice extract and dipotassium glycyrrhizate was surprisingly the most effective for maintaining the viscosity of HA gel as compared to either compound alone, and was likewise more effective as compared with other inhibitors like VcPal, Apigenin and PGA. Tannic acid, is known to be a super-efficient inhibitor of HAase, but it is not a desirable candidate for use in applications to keratinous tissue due to its potentially adverse side effects as a non-competitive inhibitor of HAase.

Formula for Inhibition Factor:

To describe the synergy clearly, the “inhibition factor,” below, reflects the extent of inhibition of HAase activity, where n=viscosity:

$$Inhibition factor ( \% ) = \frac{-}{{η}_{HA} -} \times 100 \%$$

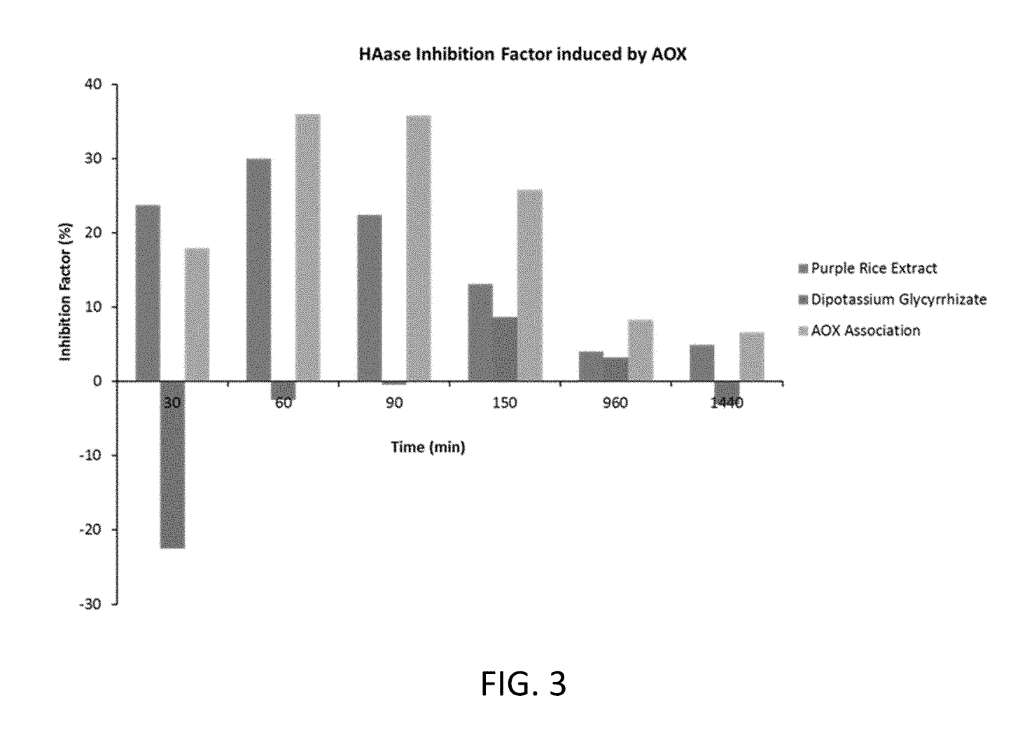

Results: Referring now to FIG. 3, the results of the determined Inhibition Factor are shown in a bar graph based on the results shown in Table 1, below. In this gel model, purple rice extract was observed to be a potent inhibitor of hyaluronidase while dipotassium glycyrrhizate showed a very mild effect. The gel test was consistent with the results of the in vitro assay, confirming that 1:1 association of the two ingredients elicited a sustained synergistic response.

TABLE 1
Demonstration of synergistic Inhibition

| | Sum of % inhibition of individual | % Inhibition of |
|---|---|---|
| Time point | compounds | Combined compounds |
| 30 min | 0.713996 | 9.994316 |
| 60 min | 21.57602 | 28.25235 |
| 90 min | 17.90779 | 29.15343 |
| 150 min | 18.52615 | 21.96708 |
| 960 min | 6.619925 | 7.523064 |
| 1440 min | 1.672876 | 6.096616 |

Exemplary formulations of compositions in accordance with the disclosure include HA, purple rice extract and dipotassium glycyrrhizate. Other ingredients may also be present as representative of various forms of formulations for application to keratinous tissue, for example skin. Thus, in some exemplary embodiments, formulations made according to the disclosure are shown in Table 2.

TABLE 2
Inventive Formulations

| Ingredient | Ex. 1 | Ex. 2 | Ex. 3 |
|---|---|---|---|
| WATER | q.s. | q.s. | q.s. |
| GLYCERIN | 5.00 | 5.00 | 5.00 |
| EMULSIFIER | 0.9 | 0.9 | 1 |
| PRESERVATIVE | 0.90 | 0.90 | 0.90 |
| DIPOTASSIUM GLYCYRRHIZATE | 2.00 | 2.00 | 2.00 |
| HYALURONIC ACID | 1.2 | 1.1 | 0.65 |
| DEXTRIN (and) ORYZA SATIVA (RICE) | 1.20 | 1.20 | 1.20 |
| EXTRACT | | | |
| THICKENER | 0.60 | 0.60 | 0.60 |
| SODIUM HYDROXIDE | 0.045 | 0.045 | 0.045 |
| ORGANIC SOLVENT | 5.50 | 5.50 | 6.50 |
| SILICONE POLYMER | 16.00 | 16.00 | 16.00 |

Raw Materials

Dextrin (and) oryza sativa (rice) extract includes dextrin having 50% active ingredient, and purple rice extract-pc having 50% active ingredients, from Oryza Oil & Fat Chemical. Dipotassium glycyrrhizate, having 100% active ingredient, from Maruzen. Sodium hyaluronate, having 100% active ingredient, from Soliance (Givaudan)/bloomage Freda Biopharm/sochibios/shandong Topscience biotech. Sodium hyaluronate, having 100% active ingredient, from Soliance (Givaudan).

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An hyaluronic acid adjuvant composition comprising:
(a) hyaluronic acid
(b) Purple rice extract; and
(c) Dipotassium glycyrrhizate;
wherein said hyaluronic acid (a), and purple rice extract (b), and dipotassium glycyrrhizate (c), are present in amounts, when combined, sufficient to produce synergistic anti-hyaluronidase activity.

2. A composition according to claim 1, comprising at least one additional hyaluronidase inhibitor selected from the group consisting of cucumis sativus fruit, terminalia catappa, ascorbic acid, mushroom stem extract, ganoderma cucidum extract and combinations thereof.

3. A composition according to claim 1, wherein one or more of HA, purple rice extract, and dipotassium glycyrrhizate is present in solid or semi-solid soluble or swellable form selected from nano-needles, microneedles, and combinations of these.

4. A composition according to claim 3, wherein HA is present in the form of microneedles.

5. A composition according to claim 1, further comprising and one or more components for forming one of an aqueous serum, an oil-in-water emulsion, and a water-in-oil emulsion.

6. A composition according to claim 1, wherein the composition is for topical application, comprising:
(a) hyaluronic acid is present up to about 3%;
(b) purple rice extract is present up to about 3%; and
(c) dipotassium glycyrrhizate is present up to about 5%.

7. A composition according to claim 1, wherein the composition is for topical application, comprising:
(a) hyaluronic acid is present from about 1.3%;
(b) purple rice extract is present from about 1.2%; and
(c) dipotassium glycyrrhizate is present from about 2%.

8. A composition according to claim 1, wherein the composition is for injection, comprising:
(a) hyaluronic acid present from about 0.01% up to about 3%;
(b) purple rice extract present from about 0.001% up to about 3%; and
(c) dipotassium glycyrrhizate is present from about 0.001% up to about 5%.

9. A composition according to claim 1, wherein the composition is for injection, comprising:
(a) hyaluronic acid present up to about 3%;
(b) purple rice extract present up to about 0.5%; and
(c) dipotassium glycyrrhizate present up to about 0.5%.

10. An hyaluronic acid adjuvant composition comprising:
(a) hyaluronic acid
(b) Purple rice extract; and
(c) Dipotassium glycyrrhizate;
wherein said hyaluronic acid (a), and purple rice extract (b), and dipotassium glycyrrhizate (c), are present in amounts, when combined, sufficient to produce synergistic anti-hyaluronidase activity, and
wherein one or more of HA, purple rice extract, and dipotassium glycyrrhizate is present in solid or semi-solid soluble or swellable form selected from nano-needles, microneedles, and combinations of these.

11. A composition according to claim 11, wherein HA is present in the form of microneedles.

12. A composition according to claim 11, comprising
(a) hyaluronic acid present from about 40% up to about 100%;
(b) purple rice extract present from about 0.001% up to about 3%; and
(c) dipotassium glycyrrhizate is present from about 0.001% up to about 5%.

13. A composition according to claim 11, comprising
(a) hyaluronic acid present from about 80% up to about 100%;
(b) purple rice extract present from about 0.001% up to about 0.5%; and
(c) dipotassium glycyrrhizate is present from about 0.001% up to about 0.5%.

14. A composition according to claim 11, comprising
(a) hyaluronic acid present up to about 100%;
(b) purple rice extract present up to about 0.5%; and
(c) dipotassium glycyrrhizate present up to about 0.5%.

15. A composition according to claim 12, wherein purple rice extract, and dipotassium glycyrrhizate are present in one of fluid, solid, and semi-solid soluble or swellable form.

16. A method for providing an HA adjuvant to keratinous tissue, comprising:
providing a composition according to claim 1 for one or a combination of topical application and injectable application to skin.

17. A method according to claim 16, wherein the composition is provided for injectable application.

18. A method according to claim 16, wherein the composition is provided for injectable application of purple rice extract and dipotassium glycyrrhizate, and topical application of HA.

19. A method for providing an HA adjuvant to keratinous tissue, comprising:
providing a composition according to claim 12 for topical application to skin.

20. A method for providing an HA adjuvant to keratinous tissue, comprising:
providing a composition according to claim 12 comprising HA microneedles for topical application and purple rice extract and dipotassium glycyrrhizate for injectable application.
